(19) United States
(12) Patent Application Publication
(10) Pub. No.: US 2025/0084378 A1
(43) Pub. Date: Mar. 13, 2025

(54) NONHUMAN STEM CELLS AND THEIR USE FOR PRODUCTION OF CULTURED MEAT

(71) Applicant: ATELIER MEATS CORP., Calgary (CA)

(72) Inventors: Maghsoud DARIANI, Fanwood, NJ (US); Mohit BHATIA, Manalapan, NJ (US)

(21) Appl. No.: 18/723,927

(22) PCT Filed: Dec. 20, 2022
(86) PCT No.: PCT/US2022/053447

§ 371 Date: Jun. 25, 2024

Related U.S. Application Data
(60) Provisional application No. 63/295,114, filed on Dec. 30, 2021.

(51) Int. Cl. C12N 5/077

(52) U.S. Cl. CPC C12N 5/0653; C12N 2506/02; C12N 2509/00

(57) ABSTRACT

Methods for production of nonhuman mammalian cultured meat fit for human consumption involving co-culturing cells isolated from nonhuman mammalian umbilical cord and/or placenta and/or components thereof with extracellular matrix (ECM) derived from nonhuman mammalian placentas and products produced by these methods are provided.

This patent application claims the benefit of priority from U.S. Provisional Application Ser. No. 63/295,114, filed Dec. 30, 2021, teachings of which are herein incorporated by reference in their entirety.

### FIELD

The present disclosure relates to methods for the isolation, culture expansion and differentiation of nonhuman mammalian umbilical cord and placenta derived stem cells, compositions of these cells, and methods for use of these cells in production of cultured meat.

### BACKGROUND OF INVENTION

As it has been shown for human placentas and the attached umbilical cord, these tissues are known to contain several types of stems cells (Weiss and Troyer, 2006). These cells are characterized by their ability to differentiate into other cell types such as adipocytes (fat cells), chondrocytes (cartilage cells), myoblasts (muscle cells), etc. A pluripotent stem cell is able to differentiate into all 3 primary germ layers including ectoderm, mesoderm and endoderm. These stem cells are also characterized by certain cell surface markers including CD10, CD29, CD44, CD105 and others (Weiss and Troyers, 2006). Like humans, mammals such as cows and pigs also deliver a placenta and umbilical cord along with the calf and the piglet. As such, similar umbilical cord stem cells have also been identified in bovine placentas (Raoufi et al, 2010). Xiong et al (2014) have isolated bovine umbilical cord mesenchymal stem cells (UCMSCs) expressing genes for CD29, CD44, CD73, CD90, and CD166. In a separate study, Cardoso et al (2012) isolated stem cells with cell surface markers including CD105+, CD29+, CD73+ and CD90+.

### SUMMARY

This disclosure focuses on unique methods for isolation of nonhuman mammalian multi-potent cells, culture expansion methods to maintain cell surface phenotype and differentiation methods to adipocytes (fat cells), muscle cells, cartilage and endothelial vascular cells. Further, this disclosure includes combining or co-culturing the culture expanded and differentiated cells with a nonhuman mammalian derived placental extracellular matrix or ECM to generate a combination product that is fit for human consumption.

An aspect of this disclosure relates to methods of isolation of stem cells from nonhuman mammalian umbilical cord, placenta and vasculature and compositions of those cell types as defined by cell surface markers.

Another aspect of this disclosure relates to methods for producing the compositions of the nonhuman mammalian placental and umbilical cord cells after cell culture expansion as defined by their phenotype (surface markers).

Another aspect of this disclosure relates to methods for differentiation of the expanded cells into adipocytes (fat cells), myocytes (muscle cells), chondrocytes (cartilage) and endothelial cells (blood vessel) and other cell types critical to formation of cultured meat.

Yet another aspect of this disclosure relates to use of the differentiated cells and culturing the cells on decellularized nonhuman mammalian placenta scaffolds thereof in production of nonhuman mammalian cultured meat for consumption.

### BRIEF DESCRIPTION OF THE FIGURES

FIGS. 1A and 1B are phase contrast images of bovine umbilical cord stromal cells (BUSC) isolated from bovine umbilical cord tissues (P0). Representative images for the cell fraction BUSC (C) (FIG. 1A) and tissue fraction BUSC (T) (FIG. 1B) are shown. The scale bar is equal to 100 μm.

FIGS. 2A, 2B, 2C and 2D are phase contrast images of BUSC cells at different passages. Representative images of BUSC at different passages (P0 (FIG. 2A), P1 (FIG. 2B), P2 (FIG. 2C) and P4 (FIG. 2D)) are shown. The scale bar is equal to 100 μm.

FIG. 3 provides representative graphs of flow cytometry analyses with antibodies against human antigens, which showed cross reactivity to bovine cells.

FIG. 4 shows representative images from oil red O staining of human mesenchymal stem cells (hMSCs) and BUSC cells after 12 days of adipogenic differentiation. 2×10⁴ of cells were seeded to each well of 24-well plate and induced to differentiate for 12 days. Representative images are shown. The scale bar is equal to 50 μm.

### DETAILED DESCRIPTION

Various embodiments and aspects of the inventions will be described with reference to details discussed below and will illustrate the various embodiments. The following description of the invention is not to be construed as limiting the invention. Numerous specific details are described to provide a thorough understanding of various embodiments of the present invention. However, in certain instances, well-known or conventional details are not described in order to provide a concise discussion of embodiments of the present inventions. Reference in the specification to “one embodiment” or “an embodiment” or “another embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

This invention relates to compositions comprising stem cells and use of such stem cells isolated from nonhuman mammalian umbilical cords and related tissues such as, but not limited to, Wharton's Jelly, placenta decidua and placental vasculature, for the generation of cultivated meat fit for human consumption.

In one nonlimiting embodiment, the cells are isolated from the umbilical cord, the placenta and/or its vasculature of a nonhuman mammal such as, but not limited to, bovine (cow or buffalo), porcine (pig), goats, and sheep.

In one nonlimiting embodiment, multi-potent stem cells are isolated from the umbilical cord of the nonhuman mammal.

In another nonlimiting embodiment, the cells are taken from the umbilical cord and Wharton's Jelly. Wharton's Jelly is contained within the umbilical cord.

In another nonlimiting embodiment, the cells are taken from the placental decidua and the placental vasculature.

The multi-potent stem cells are culture expanded while maintaining their cell surface markers (phenotype).

In one nonlimiting embodiment, the cells show a cell surface phenotype, which includes one or more of the following markers CD10+, CD13+, CD29+, CD44+, CD73+, CD90+, CD105+ or CD200+.

Cells are culture expanded until the cell surface markers begin to alter or the cells achieve senescence or the loss of power to grow and divide. In one nonlimiting embodiment, the phenotype is maintained through 10 or more doublings or expansions.

In one nonlimiting embodiment, the isolated stem cells are culture expanded through several passages to senescence.

In various embodiments, the culture-expanded cells are differentiated into smooth muscle cell precursors, adipocytes, endothelial cells and chondrocytes.

In one nonlimiting embodiment, the cell culture expanded cells are differentiated into various cells representing the 3 germ layers ectoderm, endoderm and mesoderm. These cells include adipocytes, myocytes (or muscle precursors), endothelial (blood vessel) and chondrocytes (cartilage).

These cells are cultured to sufficient densities to create cell banks for later seeding onto scaffolds as disclosed herein.

In one nonlimiting embodiment, the cell culture expanded stem cells are used to create master cell banks for storage at low temperature <−80° C.).

In one nonlimiting embodiment, the differentiated cells are used to create master cell banks for storage at low temperature <−80° C.).

In one nonlimiting embodiment, the cells are co-cultured with decellularized nonhuman mammalian placental extracellular matrix ECM to create a cultured meat product that is fit for human consumption. A nonlimiting example of a matrix ECM which can be used is that described in PCT/US2022/048680 filed Nov. 2, 2022, teachings of which are herein incorporated by reference in their entirety.

In one nonlimiting embodiment, the cultured meat is created by co-culturing the adipocytes, chondrocytes, muscle cells and endothelial cells at the same time.

In one nonlimiting embodiment, the cultured meat is created by co-culturing the adipocytes, chondrocytes, muscle cells and endothelial cells in a specific order to generate a specific structure for the meat.

In one nonlimiting embodiment, the cultured meat is created by co-culturing the adipocytes, chondrocytes, muscle cells and endothelial cells in different amounts to create cultured meats of different fat, protein content or physical structure.

The nonhuman mammalian placental stem cells referred to here is meant to encompass all stem cells derived from the placenta and its components, the umbilical cord, cord blood, Wharton's Jelly, the placenta decidua and the vasculature of the placenta.

The following nonlimiting examples are provided to further illustrate the present invention.

### EXAMPLES

### Example 1. Isolation of Bovine Umbilical Cord Stromal Cells

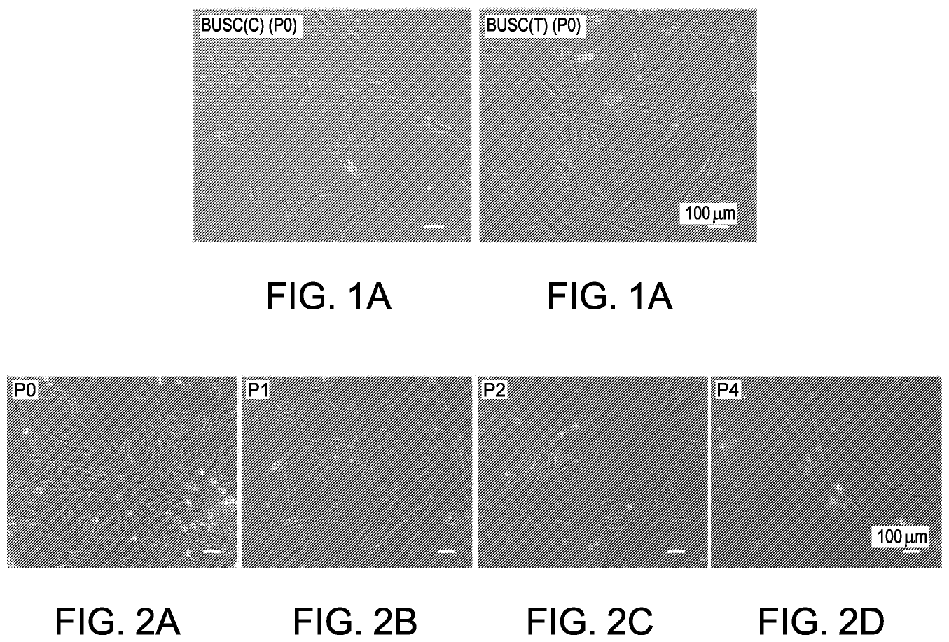

Bovine umbilical cord stromal cells (BUSC) were isolated from fresh or cryopreserved bovine umbilical tissues following a procedure modified and improved from procedures of out-migration method and enzymatic digestion method as described by set forth by Xiong et al. (Anim Cells Syst 2014 18 (1): 59-67), Cardoso et al. (Bmc Biotechnol 2012 12:18); and Wang et al. Stem Cells 2004 22 (7): 1330-7. Specifically, umbilical cord tissues were disinfected in 70% ethanol for 2-3 minutes followed by washing with phosphate buffered saline (PBS). The surrounding connective tissues were trimmed away from the disinfected tissues. Tissues were cut into 2-5 mm in length segments and digested in digestion solution (MEM-α complete medium+1× Antibiotic-Antimycotic+1 mg/mL of collagenase type I (Worthington Biochemical Corporation Cat #LS004196, Code: CLS-1, Lot #40N205980)) at 5 mL/gram of tissue. The digestion was incubated in the cell culture incubator with gentle rotation for 3 hours. After digestion, an equal volume of PBS was added to the digestion mixture and the mixture was passed through a 45 μm pore cell strainer. The fraction passing through the strainer is referred as “cell fraction” and the undigested tissues are referred as “tissue fraction”. Both cell and tissue fractions were washed using excess volumes of PBS. The cell fraction BUSC (C) and tissue fraction BUSC (T) were then cultured in 100 mm cell culture dishes for 7-10 days (P0) (see FIGS. 1A and 1B, respectively). The yield of cells from umbilical cord tissues on Day 7 was about 7×10⁶ cells/g of wet tissue. Cells from the cell fraction and tissue fraction were then cryopreserved as BUSC (C) P1 and BUSC (T) P1, respectively.

### Example 2. In Vitro Expansion (Culture) of Bovine Umbilical Cord Stromal Cells

P1 cells (20×10⁴) were seeded onto one 100 mm cell culture dish in growth medium (MEM-α base medium+10% fetal bovine serum (FBS)+1× Antibiotic-Antimycotic) and incubated at 37° C. in an incubator with 5% CO₂ and 90% humidity until reaching 85% confluence. Once cells reached 85% confluence, cells were trypsinized with 1 mL trypsin (Gibco), neutralized with 4 ml of cell growth medium and then the number of P2 cells were counted using a hemocytometer. To set up the next passage, 20×10⁴ cells of P2 cells were seeded onto one 100 mm dish and cultured until 85% confluence. This process was repeated and cell numbers at the end of each passage were recorded. The morphologies of cells at different passages are shown in FIGS. 2A through 2D.

The doubling time was calculated as follows:

Doubling time (h)=Duration (h)×ln(2)/ln(Final cell number/Initial cell number).

The fold of amplification was calculated as follows: Final cell number/Initial cell number.

The expansion of BUSC from different batches was monitored up to passage 6 and results are shown in Table 1.

TABLE 1

| | | | | Doub- | | | | | | Doub- | | | | | | Doub- | |
|---|---|---|---|---|---|---|---|---|---|---|---|---|---|---|---|---|---|
| | | | | ling | Ampli- | | | | | ling | Ampli- | | | | | ling | Ampli- |
| | | | | time | fication | | | | | time | fication | | | | | time | fication |
| 1BUSC | Start | End | Days | (h) | (fold) | 2BUSC | Start | End | Days | (h) | (fold) | 3BUSC | Start | End | Days | (h) | (fold) |
| | P2 | P3 | | | | | P2 | P3 | | | | | P2 | P3 | | | |
| Cells | 8 | 118 | 9 | 56 | 15 | Cells | 20 | 168 | 7 | 55 | 8 | Cells | 20 | 142.5 | 7 | 59 | 7 |
| (×10\hat{ }4) | | | | | | (×10\hat{ }4) | | | | | | (×10\hat{ }4) | | | | | |
| | P3 | P4 | | | | | P3 | P4 | | | | | P2 | P3 | | | |
| Cells | 18 | 328 | 6 | 34 | 18 | Cells | 20 | 45 | 7 | 144 | 2 | Cells | 20 | 123.8 | 7 | 64 | 6 |
| (×10\hat{ }4) | | | | | | (×10\hat{ }4) | | | | | | (×10\hat{ }4) | | | | | |
| | P4 | P5 | | | | | P4 | P5 | | | | | P3 | P4 | | | |
| Cells | 82 | 254 | 7 | 103 | 8 | Cells | 20 | 40 | 12 | 288 | 2 | Cells | 20 | 104 | 11 | 111 | 5.2 |
| (×10\hat{ }4) | | | | | | (×10\hat{ }4) | | | | | | (×10\hat{ }4) | | | | | |
| | P5 | P6 | | | | | P5 | P6 | | | | | P5 | P6 | | | |
| Cells | 20 | 243.5 | 12 | 80 | 8 | Cells | 20 | 36 | 8 | 226 | 2 | Cells | 20 | 92.5 | 10 | 100 | 4.6 |
| (×10\hat{ }4) | | | | | | (×10\hat{ }4) | | | | | | (×10\hat{ }4) | | | | | |

### Example 3. Analysis of Stem Cell Markers on BUSC Using Flow Cytometry

Cells were cultured in 100 mm cell culture dishes to 85% confluence and detached using 1 mL/dish of TrypLE (Tryple Express Enzyme (1×) Phenol Red, Fisher Scientific, Cat #12-605-010). About 50×10⁴ cells were resuspended in 100 μL of staining buffer (1×PBS+5% FBS+0.02% NaN₃). Individual or multiple antibodies (as listed in Table 2) were added to the cells at 5 μL of each antibody per 1×10⁶ cells. The cells were incubated with antibodies in the dark for 30 minutes followed by centrifugation at 1000 rpm for 5 minutes. The supernatant was removed and the cells were washed with 1 mL of PBS and then centrifuged again at 1000 rpm for 5 minutes. Following this second centrifugation step, the supernatant and the cells were resuspended cells in staining buffer at 20-50×10⁴ cells in 200-300 μL of staining buffer. The cells were kept on ice and flow cytometry analysis was performed using Gallios Flow Cytometer (Beckman Coulter).

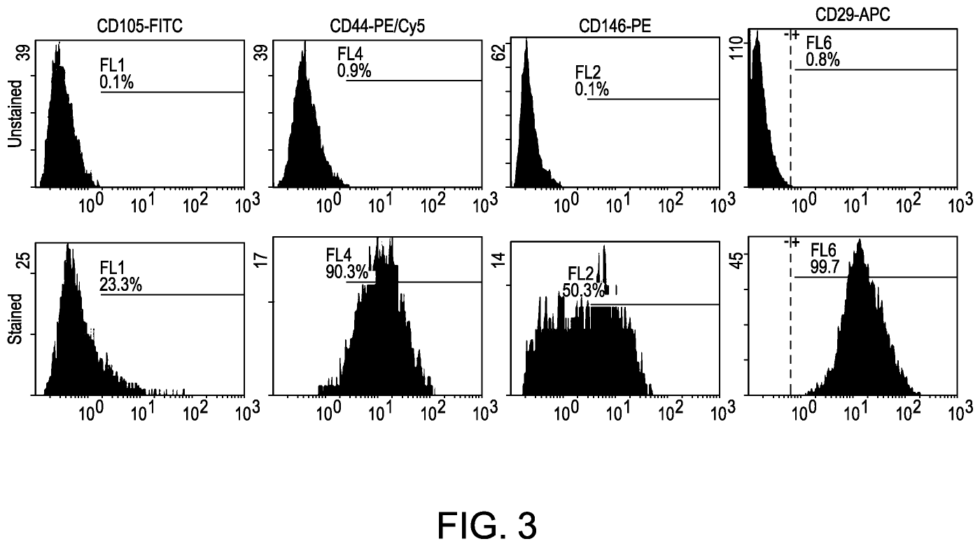

Since the antibodies against stem cell markers are developed against human antigens, the cross reactivities of those antibodies to bovine cells must be determined. The commonly used cell surface markers for human stem cells are listed in Table 2. Among all the antibodies (conjugated with fluorophores) tested, CD105-FITC, CD44-PE/Cy5, CD146-PE and CD-APC showed positive staining on bovine cells (see FIG. 3). Flow cytometry analyses indicated that the isolated BUSC cells contained at least a population of expressing mesenchymal stem cell markers.

TABLE 2

| | | | | | | | Worked |
|---|---|---|---|---|---|---|---|
| | | | | | In | | with |
| | | | Marker | | Stem | | Bovine |
| Antibodies | Antigen | Conjugates | For | Clone | Cells | Vendor | Cells |
| CD45 | Lymphocyte | FITC | Hematopoietic | HI30 | − | BioLegend | Yes |
| | common | | cells | | | | |
| | antigen | | | | | | |
| CD29 | Integrin β1 | APC | stromal and | TS2/16 | + | BioLegend | Yes |
| | | | myo/epithelial | | | | |
| | | | cells | | | | |
| CD73 | Ecto-5′- | Pacific | Immune cells | AD2 | + | BioLegend | NO |
| | nucleotidase | Blue ™ | & | | | | |
| | | | mesenchymal | | | | |
| | | | stem cells | | | | |
| CD90 | Thymocyte | PE | Thymocytes, | 5.00E+10 | + | BioLegend | No |
| | antigen | | various stem | | | | |
| | (Thy-1) | | cells, neurons | | | | |
| CD105 | Endoglin | PE | Common | 5N6 | + | Biogems | No |
| | | | marker for | | | | |
| | | | mesenchymal | | | | |
| | | | stem cells | | | | |
| CD146 | glycoprotein | PE | Endothelial | P1H12 | + | Biogems | Yes |
| | MUC18 | | cells and | | | | |
| | | | subset of | | | | |
| | | | stem cells | | | | |
| CD271 | Low-affinity | PERCP- | Stem cells | C40-1457 | + | BD | No |
| | Nerve Growth | CY5.5 | | | | | |
| | Factor | | | | | | |
| | Receptor | | | | | | |
| CD44 | transmembrane | PE-Cy5 | Stem cells and | IM7 | + | Biogems | Yes |
| | glycoprotein | | cancer stem | | | | |
| | | | cells | | | | |
| CD105 | Endoglin | FITC | Common | Polyclonal | + | Biorbyt | Yes |
| | | | marker for | orb257051 | | | |
| | | | MSC1 | | | | |

### Example 4. Adipogenic Differentiation of Bovine Umbilical Cord Stromal Cells

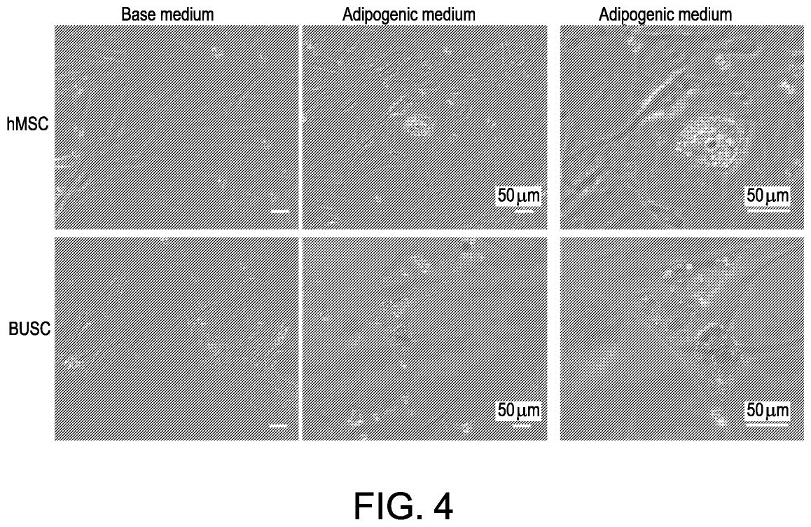

BUSC cells were cultured to 75% confluence and trypsinized and counted. 2×10⁴/well of BUSC were seeded to the wells of a 24-well plate. Human bone marrow derived mesenchymal stem cells (hMSC) were used as a positive control for adipogenic differentiation. After incubation overnight, cells were induced to adipogenic differentiation using adipogenic medium as described by Lee and Fried (Methods Enzymol 2014 538:49-65). Medium was changed every three days for 12 days. After differentiation, cells were stained with Oil Red O in accordance with the procedure described by Ghoniem et al. Anat Cell Biol 2015 48 (2): 85-94. Results depicted in FIG. 4 are indicative of the stromal cells isolated from bovine umbilical cord tissues being stem cells which can be differentiated into adipocytes.

1. A method for production of nonhuman mammalian cultured meat fit for human consumption, said method comprising co-culturing cells isolated from nonhuman mammalian umbilical cord and/or placenta and/or components thereof with extracellular matrix (ECM) derived from nonhuman mammalian placentas.

2. The method of claim 1 wherein said cells are derived from stem cells isolated from nonhuman mammalian placenta, umbilical cord, Wharton's Jelly, placental decidua and/or vasculature thereof.

3. The method of claim 2 wherein said stem cells possess at least one surface marker selected from CD10+, CD13+, CD29+, CD44+, CD73+, CD90+, CD105+ and CD200+.

4. The method of claim 3 wherein the phenotype of said stem cells is maintained through 10 or more doublings or expansions.

5. The method of claim 2 wherein the stem cells are expanded and frozen as a master cell bank prior to co-culturing.

6. The method of claim 2 wherein the stem cells are differentiated into cells of one or more germ layers selected from ectoderm, endoderm and mesoderm.

7. The method of claim 2 wherein the stem cells are differentiated into adipocytes, chondrocytes, endothelial cells and muscle precursors.

8. The method of claim 6 wherein different cell types are co-cultured together.

9. The method of claim 6 wherein the different cell types are cultured with the ECM individually in a specific order.

10. The method of claim 6 wherein the ECM tissue is cultured with different amounts of cells to affect fat, protein content and/or texture of the cultured meat.

11. The method of claim 1 wherein the cells are isolated from a nonhuman mammal selected from bovine, porcine, goat or sheep.

12. A nonhuman mammalian cultured meat fit for consumption produced in accordance with claim 1.

13. The method of claim 7 wherein different cell types are co-cultured together.

14. The method of claim 7 wherein the different cell types are cultured with the ECM individually in a specific order.

15. The method of claim 7 wherein the ECM tissue is cultured with different amounts of cells to affect fat, protein content and/or texture of the cultured meat.
